(19) United States
(12) Patent Application Publication
(10) Pub. No.: US 2005/0048022 A1
(43) Pub. Date: Mar. 3, 2005

(54) Composition comprising at least one conductive polymer and at least one film-forming polymer, and process for use thereof

(76) Inventors: Isabelle Rollat-Corvol, Paris (FR); Henri Samain, Bievres (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/892,344

(22) Filed: Jul. 16, 2004

Related U.S. Application Data
(60) Provisional application No. 60/492,296, filed on Aug. 5, 2003.

(30) Foreign Application Priority Data
Jul. 16, 2003 (FR) 03 08671

(51) Int. Cl. A61K 7/06; A61K 7/11

(52) U.S. Cl. 424/70.17

(57) ABSTRACT

A composition comprising, in a cosmetically acceptable medium, (a) at least one film-forming polymer and (b) at least one conductive polymer, for example, comprising at least one repeating unit chosen from aniline, pyrrole, thiophene, bisthiophene, furan, para-phenylene sulfide, vinylene para-phenylene, indole, aromatic amide, aromatic hydrazide, aromatic azomethine, and aromatic esters. A process using such a composition, and also to the use of this composition as a hair product base for shaping and/or holding the hairstyle. Also the use of this composition to give keratin fibers at least one optical effect.

This application claims benefit of U.S. Provisional Application No. 60/492,296, filed Aug. 5, 2003.

Disclosed herein is a composition comprising, in a cosmetically acceptable medium, at least one conductive polymer and at least one film-forming polymer. Further disclosed herein is a process for treating keratin fibers using the above-mentioned composition, and the use of this composition as a base for hair styling and/or fixing products. Also disclosed herein is the use of such a composition to impart at least one optical effect on keratin fibers.

The present disclosure relates to the field of treating keratin fibers, for example, human keratin fibers, such as hair.

Most of treatments applied to keratin fibers, such as dyeing, bleaching or permanent-reshaping processes, may have major consequences on the characteristics of fibers, such as their sheen. For example, following repeated treatments, it is not uncommon to find that the treated fibers may become somewhat dull, despite improvements that have been made in the processes used.

In order to compensate for at least some of these negative effects, and to also give hair sheen, it is known practice to use, for example, lubricating hydrophobic substances, such as organic oils, waxes, and silicones. However, the sheen effect obtained may lack intensity and may give the fibers an artificial look. Further, these compositions may give the fibers an undesirable greasy or tacky feel.

In one embodiment, the compositions disclosed herein may give treated keratin fibers a sheen appearance without encountering at least some of the drawbacks encountered with prior art compositions.

Moreover, in some embodiments, when it is applied to keratin fibers, the composition disclosed herein may give color to the fibers.

Disclosed herein is a composition comprising, in a cosmetically acceptable medium:
(a) at least one film-forming polymer and
(b) at least one conductive polymer.

Further disclosed herein is a process for treating human keratin fibers, such as hair, comprising,
applying, to wet or dry fibers, at least one composition comprising, in a cosmetically acceptable medium:
(a) at least one film-forming polymer and
(b) at least one conductive polymer; and
drying the fibers or leaving the fibers to dry.

Also disclosed herein is use of the composition as a hair product base for shaping and/or holding the hairstyle.

Also disclosed herein is the use of a composition comprising at least one film-forming polymer and at least one conductive polymer, to impart at least one optical effect on keratin fibers.

For example, in one embodiment, the composition disclosed herein may uniformly give the fibers, for example, a sheen that is at least one of more intense, more natural and more aesthetic than with the means of the prior art.

Moreover, when the at least one conductive polymer, present in the composition disclosed herein, absorbs in the visible spectrum, at least one optical effect, for example, chosen from sheen and color may be obtained simultaneously.

Also fibers treated with the composition disclosed herein may have a soft, non-greasy feel.

In the text hereinbelow and unless otherwise indicated, the limits of a range of values are understood as forming part of that range.

As used herein, the term “optical effect” means sheen, color, metallic, goniochromatic, moiré, fluorescent, thermochromic, and electrochromic effects.

Moreover, it should be noted that the sheen corresponds to the light intensity reflected at an angle α when the lock of hair is illuminated under an angle −α. The angle α used to measure this specular reflection, in other words the sheen, is equal to 20°. This provision of sheen is measured using a glossmeter as described in ISO standard 2813-1994 from AFNOR (August 1994, amended February 1997).

Conductive Polymers

As used herein, the term “conductive polymer” means a molecular structure in which the monomer(s) has (have) high electron delocalization and whose arrangement in the polymer skeleton allows the π orbitals to overlap. This chemical characteristic is reflected by electrical conduction, which may or may not be accompanied by absorption in the UV-visible spectrum, or even in the infrared spectrum.

As used herein, the expression “conductive polymer absorbing in the visible spectrum” means any conductive polymer having a non-zero absorbance in the wavelength ranging from 400 to 800 nm, even if the absorption maxima of the polymer is outside this range.

The at least one conductive polymer used in the composition disclosed herein is chosen from conductive polymers that are soluble or dispersible in the cosmetic medium suitable for use.

The at least one conductive polymer is soluble in the medium when it forms an isotropic clear liquid at 25° C. in the medium comprising water or mixtures of water and at least one solvent, wherein the solubility is obtained throughout all or part of a concentration ranging from 0.01% to 50% by weight of the at least one conductive polymer.

In one embodiment, the at least one conductive polymer is chosen from conductive polymers that are soluble or dispersible in an aqueous medium, for example, in water.

The at least one conductive polymer is dispersible in a medium comprising water or mixtures of water and at least one solvent if, at 0.01% by weight, at 25° C., it forms a stable suspension of fine, generally spherical particles. The mean size of the particles constituting the dispersion is less than 1 μm, for example, ranging from 5 to 400 nm and, further, for example, from 10 to 250 nm. These particle sizes are measured by light scattering.

It should be noted, that in some embodiments, the at least one conductive polymer does not require the use of a dispersant.

The at least one conductive polymer may, for example, be in a form that is soluble in the medium of the composition.

Further, the at least one conductive polymer may have a conductivity ranging from 1×10⁻⁵ to 5×10⁵ siemens/cm, further, for example, from 1×10⁻³ to 1×10⁵ siemens/cm and, even further, for example, from 1×10⁻¹ to 1×10⁴ siemens/cm.

The conductivity is measured using a current generator (RM2 Test Unit sold by the company Jandel) equipped with a four-point measuring head (Universal four-point probes sold by the company Jandel). The four points, aligned and separated by the same space d, are applied by simple pressure to the sample to be analysed. A current I is injected via the outer points using the current source, thus creating a variation in potential. The voltage U is measured between the two inner points connected to the voltmeter of the current generator.

In this configuration, the conductivity of the sample expressed in S/cm is given by the following expression:
σ=(K×I)/(U×e)
wherein:
   K is a coefficient depending on the position of the contacts on the surface of the sample. When the points are aligned and equidistant,
   K is equal to: π/log(2);
   I is the value of the injected current, expressed in amperes;
   U is the measured voltage value, expressed in volts; and
   e is the thickness of the sample, expressed in cm.

This expression can be used only when the thickness of the material is negligible compared with the distance d existing between two points (e/d<0.25). In order to obtain sufficiently small thicknesses and thus to be able to calculate the conductivity of the material, it is recommended to perform the measurement on a non-conductive support (for example a glass slide) coated with the material to be analyzed, obtained by evaporation of a dilute solution. In order to improve the homogeneity of the coating to be analysed, it is also recommended to use the deposition technique known as spin coating.

According to one embodiment, the at least one conductive polymer present in the composition is chosen from polymers comprising at least one repeating unit of the following formulae:
anilines of formula (I) below: 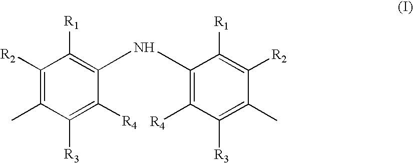
pyrroles of formulae (IIa) and (IIB) below: 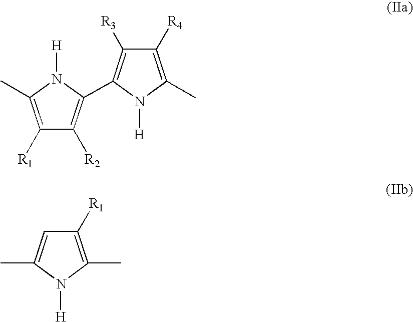
thiophenes and bisthiophenes of formulae (IIIa), (IIIb) and (IIIc) below: 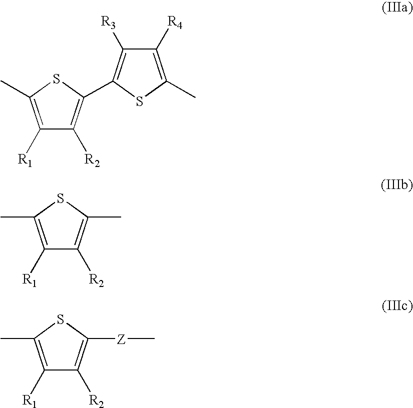
furans of formula (IV) below: 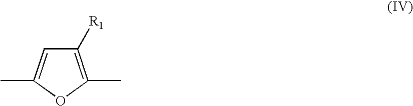
para-phenylene sulfides of structure (V) below: 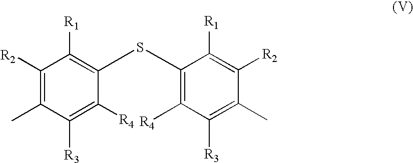
para-phenylenevinylenes of formula (VI) below: 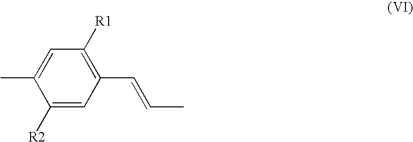
indoles of formula (VII) below: 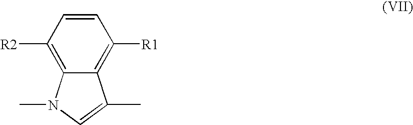
aromatic amides of formulae (VIIIa), (VIIIb), (VIIIc) and (VIIId) below: 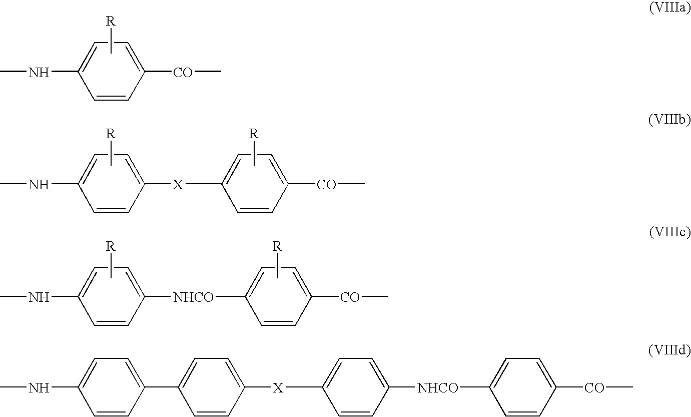
aromatic hydrazides of formulae (IXa), (IXb) and (IXc) below: 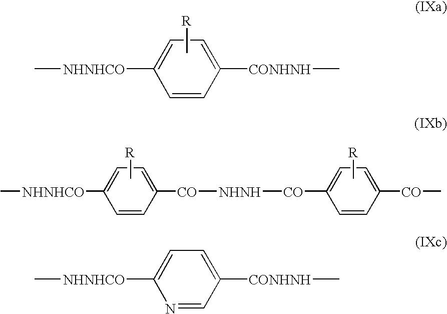
aromatic azomethines of formulae (Xa), (Xb) and (Xc) below: 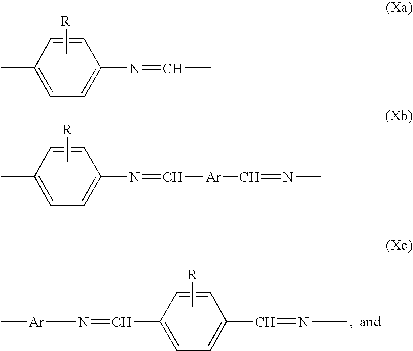
and
aromatic esters of formulae (XIa), (XIb) and (XIc) below: 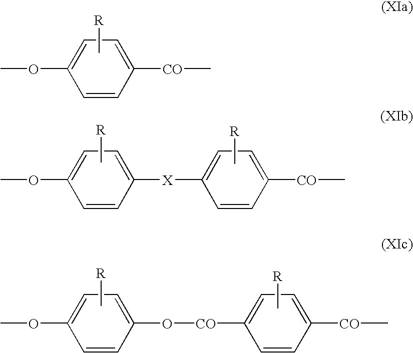
wherein, in formulae (I) to (XI):
   R, R1, R2, R3, and R4, which may be identical or different, are each chosen from a hydrogen atom, —R′, —OR′, —COOR′, and —OCOR′, wherein R′ is chosen from linear and branched C₁-C₂₀ alkyl radicals, halogen atoms, nitro radicals, cyano radicals, cyanoalkyl radicals, solubilizing groups, and solubilizing groups comprising a spacer group that bonds to the ring;
   Ar is chosen from radicals comprising at least one radical chosen from monoaromatic and polyaromatic radicals;
   X is chosen from —NHCO—, —O—, —S—, —SO₂—, —N═N—, —C(CH₃)₂—, —CH₂—, —CH═CH—, and —CH═N—; and
   Z is chosen from —CH═CH— and —C≡C—.

In one embodiment, Ar is chosen from radicals comprising at least one radical chosen from the following:

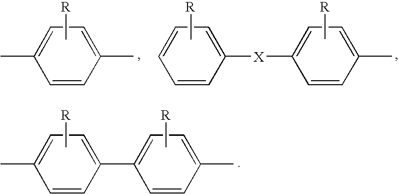

As used herein, the term “solubilizing group” means a group that ensures the dissolution of the molecule in the cosmetic medium, such that the at least one conductive polymer has a conductive nature after drying the composition.

It is clear that the at least one conductive polymer present in the composition disclosed herein may comprise at least one repeating unit comprising at least one solubilizing group, and at least one other repeating unit lacking the at least one solubilizing group.

The solubilizing groups may, for example, be chosen from the following radicals:
carboxylic (—COOH) radicals and carboxylate (—COO-M⁺) radicals, wherein M is chosen from alkali metals, such as sodium and potassium; alkaline-earth metals; organic amines, such as primary, secondary, and tertiary amines; alkanolamines;
and amino acid radicals, sulfonic (—SO₃H) radicals and sulfonate (—SO₃⁻M⁺) radicals, M having the same definition as above;
primary, secondary, and tertiary amine radicals;
quaternary ammonium radicals, such as —N(R′)₃⁺Z⁻, wherein Z is chosen from Br, Cl, (C₁-C₄)alkyl-OSO₃ and R′, which may be identical or different, is chosen from linear and branched C₁ to C₂₀ alkyls, or two of the R's form a heterocycle with the nitrogen to which they are attached;
hydroxyl radicals; and
poly((C₂-C₃)alkylene oxide) radicals.

The carboxylic or sulfonic acid radicals may be optionally neutralized with at least one base, for example, chosen from sodium hydroxide, 2-amino-2-methylpropanol, triethylamine, and tributylamine.

The amine radicals may be optionally neutralized with at least one mineral acid, for example, chosen from hydrochloric acid and organic acids, such as acetic acid and lactic acid.

In addition, it should be noted that the solubilizing radicals may be connected to the ring via a spacer group, for example, chosen from —R″—, —OR″—, —OCOR″—, and —COOR″—, wherein R″ is chosen from linear and branched C₁-C₂₀ alkyl radicals optionally comprising at least one hetero atom, such as oxygen.

In one embodiment, the radicals R, R1, R2, R3, and R4, which may be identical or different, are each chosen from hydrogen, R′, —OR′, —OCOR′, and —COOR′, wherein R′ is chosen from linear and branched C₁-C₆ alkyl radicals, and from the following neutralized or non-neutralized solubilizing groups: —COOH, —CH₂COOH, —CH₂OH, —(CH₂)₆OH, —(CH₂)₃SO₃H, —O(CH₂)₃SO₃H, —O(CH₂)₃N(CH₂CH₃)₂, —[(CH₂)₂O]ₓCH₂CH₂OH, and —[(CH₂)₂O]ₓCH₂CH₂OCH₃, wherein x is an average number ranging from 0 to 200.

The number n of repeating units in the at least one conductive polymer may, for example, range from 5 to 10 000, for example, from 5 to 1000, even further, for example, from 10 to 1000 and, even further, for example, from 20 to 700.

In another embodiment, the at least one conductive polymer is such that at least one radical chosen from R, R1, R2, R3, and R4 is a solubilizing group.

In another embodiment, the at least one conductive polymer used comprises at least one solubilizing group per repeating unit. Thus, for example, at least one radical chosen from R, R1, R2, R3, and R4 is a solubilizing group.

In another embodiment, the at least one conductive polymer is soluble in the medium of the composition.

The at least one conductive polymer present in the composition disclosed herein may be well known to those skilled in the art and may be described, for example, in the book “Handbook of Organic Conductive Molecules and Polymers”—Wiley 1997—New York, Vol 1, 2, 3, and in the review Can. J. Chem. Vol 64, 1986.

Polythiophenes and their synthesis are, for example, described in the article taken from the review Chem. Mater. 1998, Vol. 10, No 7, pages 1990-1999 by the authors Rasmussen S. C., Pickens J. C. and Hutchison J. E. “A New, General Approach to Tuning the Properties of Functionalized Polythiophenes: The Oxidative Polymerization of Monosubstituted Bithiophenes”; in the article taken from the review Macromolecules 1998, 31, pages 933-936, by the same authors “Highly Conjugated, Water-soluble Polymers Via Direct Oxidative Polymerization of Monosubstituted Bithiophenes”. In addition to polymerization via chemical or electrochemical oxidation, the polythiophenes may also be obtained by at least one reaction chosen from polycondensation (dihalothiophene; catalysis with nickel or palladium complexes); Suzuki coupling (coupling between a halogen functional group, for example, bromine, and a boronic acid, catalysis: palladium complex and base; this then gives coupling of AA-BB type (reaction of monomers of the type A-X-A with B-X′-B) or of A-B type (reaction of several monomers of the type A-X-B); Stille coupling (formation of a carbon-carbon bond in the presence of a Pd-based catalyst—AA-BB or A-B type); Reike polymerization (organozinc in the presence of a nickel complex); and polymerization of McCulloch type, etc.

In one embodiment, the at least one conductive polymer present in the composition disclosed herein are moreover described in International Patent Publication No. WO 99/47570.

Examples of the at least one conductive polymer that are suitable for use in the composition disclosed herein include polymers corresponding to formulae (IIIa), (IIIb) and (IIIc) wherein the solubilizing groups may, for example, be chosen from carboxylic acid radicals; neutralized carboxylic acid radicals; sulfonic acid radicals; neutralized sulfonic acid radicals; tertiary amine radicals; and quaternary ammonium radicals such as —N(R′)₃⁺Z⁻, wherein Z is chosen from Br, Cl, (C₁-C₄)alkyl-OSO₃, and R′, which may be identical or different, is chosen from linear and branched C₁ to C₂₀ alkyls, or two of the R's form a heterocycle with the nitrogen to which they are attached; and wherein the solubilizing groups are optionally connected to the ring via a spacer group.

Thus, the polymerization may be performed via chemical or electrochemical oxidation of the corresponding thiophene monomer or via polycondensation.

For example, the polythiophenes of formulae (IIIa) and (IIIb) may be obtained by at least one reaction chosen from the following:
polymerization via oxidation (for example, with FeCl₃ catalysis);
polycondensation of dihalothiophene catalysed with nickel or palladium complexes (e.g.: NiCl₂(dppe)₂);
Suzuki coupling (coupling between a halogen functional group, for example, bromine, and a boronic acid, catalysis: palladium complex and base; this then gives coupling of AA-BB type (reaction of monomers of the type A-X-A with B-X′-B) or of A-B type (reaction of several monomers of the type A-X-B);
Stille coupling (formation of a carbon-carbon bond formed in the presence of a Pd-based catalyst—AA-BB or A-B type);
Reike polymerization (organozinc in the presence of a nickel complex); and
polymerization of McCulloch type, etc.

The vinylene polythiophenes of formula (IIIc), wherein Z is —CH═CH— may be obtained by at least one reaction chosen, for example, from Gilch polymerization in the presence of a strong base (potassium tert-butoxide) of 2,5-bis(bromoalkylene)thiophene; polymerization by the Wessling method via the use of a precursor based on sulfonium salts and pyrolysis; and a Wittig-Horner Wittig reaction.

The ethynylene polythiophenes of formula (IIIc) wherein Z is a —C≡C— radical may be obtained by at least one reaction chosen from Heck-Sonogashira coupling (of the type AA-BB or A-B; formation of a carbon-carbon bond between a terminal acetylenic (or true acetylenic) functional group and a bromo or iodo functional group, catalysed with a palladium/copper complex (PdCl₂(PPh₃)₃, CuI or Cu(OAc)₂) in the presence of a base such as triethylamine, diisopropyl amine, piperidine, etc.); and metathesis of alkynes in the presence of a molybdenum complex (Mo(CO)₆).

In general, the functionalization of the polythiophenes, in other words the introduction of the solubilizing or non-solubilizing group(s), is performed on the monomer before it is polymerized.

In some embodiments, the solubilizing group may be obtained after working up the polymer. This may, for example, be the case for the carboxylic acid functional group, which may be obtained by hydrolysis of the corresponding ester.

For example, in one embodiment, the solubilizing groups are chosen from carboxylic acid radicals; neutralized carboxylic acid radicals; sulfonic acid radicals; neutralized sulfonic acid radicals; tertiary amine radicals; and quaternary ammonium radicals, such as —N(R′)₃⁺Z⁻, wherein Z is chosen from Br, Cl, (C₁-C₄)alkyl-OSO₃, and R′, which may be identical or different, is chosen from linear and branched C₁-C₂₀ alkyl radicals, optionally connected to the ring via a spacer group, for example, chosen from C₁-C₂₀ alkyl radicals; and salts thereof.

According to one embodiment, the at least one conductive polymer comprises at least one repeating unit chosen from units of formulae (IIIa), (IIIb), and (IIIc), wherein at least one radical R1, R2, R3, and R4 of formula (lla) and R1 or R2 of formulae (IIIb) and (IIIc) is chosen from carboxylic acid solubilizing groups, in neutralized or non-neutralized form, optionally connected to the ring via a spacer group, for example, chosen from linear and branched C₁-C₂₀ alkyl radicals, wherein the other radical(s) is(are) hydrogen(s).

The at least one conductive polymer may be present in the composition in an amount greater than or equal to 0.001% by weight, for example, greater than or equal to 0.01% by weight, further, for example, greater than or equal to 0.1% by weight and, even further, for example, greater than or equal to 0.5% by weight, relative to the total weight of the composition. Moreover, the at least one conductive polymer may be present in an amount less than or equal to 50% by weight, for example, less than or equal to 30% by weight, further, for example, less than or equal to 20% by weight and, even further, for example, less than or equal to 10% by weight, relative to the total weight of the composition.

In another embodiment, the at least one conductive polymer is present in an amount ranging from 0.1% to 50% by weight, for example, from 0.1% to 30% by weight and, even further, for example, 0.5% to 10% by weight, relative to the total weight of the composition.

Film-Forming Polymer

As mentioned previously, the composition disclosed herein comprises at least one film-forming polymer.

For example, the at least one film-forming polymer may be chosen from cationic, anionic, amphoteric, and nonionic polymers.

In one embodiment, the at least one film-forming polymer may be soluble in the cosmetically acceptable medium or insoluble in that medium and, in this case, used in the form of dispersions of solid or liquid polymer particles (latex or pseudolatex).

The cationic film-forming polymers that may be used as the at least one film-forming polymer may be chosen, for example, from polymers comprising at least one group chosen from primary, secondary, tertiary and quaternary amine groups forming part of the polymer chain or directly attached thereto, and having a number-average molecular mass ranging from 500 to 5 000 000 and, further, for example, from 1000 to 3 000 000.

Examples of such cationic polymers include the following:
(1) homopolymers or copolymers derived from acrylic or methacrylic ester or amide derivatives and comprising at least one of the following units: 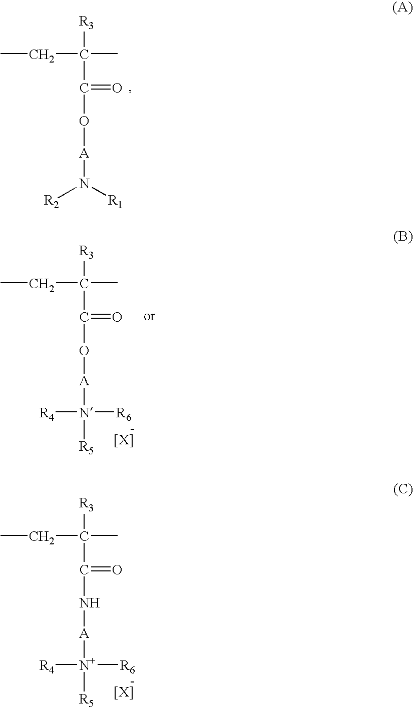
wherein:
   R₁ and R₂, which may be identical or different, are each chosen from a hydrogen atom and alkyl groups comprising from 1 to 6 carbon atoms;
   R₃ is chosen from a hydrogen atom and a CH₃ group;
   A is chosen from linear and branched alkyl groups comprising from 1 to 6 carbon atoms and hydroxyalkyl groups comprising from 1 to 4 carbon atoms;
   R₄, R₅ and R₆, which may be identical or different, are each chosen from alkyl groups comprising from 1 to 18 carbon atoms and a benzyl group; and
   X is chosen from methosulfate anions and halides, such as chloride and bromide;
(2) quaternized guar gums;
(3) quaternary vinylpyrrolidone and vinylimidazole copolymers; and
(4) chitosans and salts thereof, wherein the salts that may be used may, for example, be chosen from chitosan acetate, lactate, glutamate, gluconate, and pyrrolidonecarboxylate.

The copolymers of family (1) may, for example, also comprise at least one unit derived from comonomers that may be chosen from acrylamides; methacrylamides; diacetoneacrylamides; acrylamides; methacrylamides substituted on the nitrogen with at least one group chosen from lower (C₁₋₄) alkyl groups and groups derived from acrylic or methacrylic acids and esters thereof; vinyllactams, such as vinylpyrrolidone and vinylcaprolactam; and vinyl esters.

In one embodiment, the copolymers of family (1) may be chosen from:
copolymers of acrylamide and dimethylaminoethyl methacrylate quaternized with dimethyl sulfate or with dimethyl halide;
copolymers of acrylamide and methacryloyloxyethyltrimethylammonium chloride, which are described, for example, in Patent Application No. EP-A-080 976;
copolymers of acrylamide and methacryloyloxyethyltrimethylammonium methosulfate;
vinylpyrrolidone/dialkylaminoalkyl acrylate or methacrylate quaternized or non-quaternized copolymers, such as the products sold under the name Gafquat by the company ISP, such as Gafquat 734 and Gafquat 755 and the products named Copolymer 845, 958 and 937. These polymers are described in detail in French Patent Nos. 2 077 143 and 2 393 573;
dimethylaminoethyl methacrylate/vinylcaprolactam/vinylpyrrolidone terpolymers, such as the product sold under the name Gaffix VC 713 from the company ISP; and
vinylpyrrolidone/dimethylaminopropyl methacrylamide quaternized copolymers, for example, the product sold under the name Gafquat HS 100 by the company ISP.

Among these compounds, mention may be made of chitosan having a degree of deacetylation of 90% by weight, and chitosan pyrrolidone carboxylate sold under the name Kytamer PC by the company Amerchol.

The anionic film-forming polymers may be chosen from polymers comprising at least one group derived from carboxylic acid, sulfonic acid, and phosphoric acid and having a number-average molecular mass ranging from 500 to 5 000 000.

The carboxylic groups may, for example, be provided by unsaturated monocarboxylic or dicarboxylic acid monomers such as those corresponding to the formula: 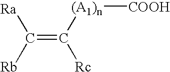
wherein n is an integer ranging from 0 to 10; A₁ is a methylene group, optionally connected to the carbon atom of the unsaturated group, or to the neighboring methylene group when n is greater than 1, via a hetero atom, such as oxygen or sulfur; Ra is chosen from a hydrogen atom and phenyl and benzyl groups; Rb is chosen from a hydrogen atom, C₁-C₄ alkyl groups, such as methyl, ethyl, and carboxyl groups; and Rc is chosen from a hydrogen atom, lower alkyl groups, —CH₂—COOH, and phenyl and benzyl groups.

The anionic film-forming polymers comprising at least one carboxylic group may be chosen, for example, from:
A) acrylic or methacrylic acid homo- and copolymers, and salts thereof and, for example, the products sold under the names Versicol E and K by the company Allied Colloid and Ultrahold by the company BASF, copolymers of acrylic acid and acrylamide, sodium salts of polyhydroxycarboxylic acids.
B) copolymers of acrylic or methacrylic acid with a monoethylenic monomer, for example, chosen from ethylene, styrene, vinyl esters, and acrylic and methacrylic acid esters, optionally grafted onto a polyalkylene glycol, such as polyethylene glycol and optionally crosslinked. Such polymers are described, for example, in French Patent No. FR-A-1 222 944 and German Patent Application No. DE-A-2 330 956, the copolymers of this type comprising at least one unit chosen from optionally N-alkylated and hydroxyalkylated acrylamide units in their chain as described, for example, in Luxembourg Patent Application Nos. 75370 and 75371. Mention may also be made of copolymers of acrylic acid and C₁-C₄ alkyl methacrylate and terpolymers of vinylpyrrolidone, acrylic acid and C₁-C₂₀ alkyl methacrylate, for example, lauryl, such as the product sold by the company ISP under the name Acrylidone LM and methacrylic acid/ethyl acrylate/tert-butyl acrylate terpolymers such as the product sold under the name Luvimer 100 P by the company BASF.
C) copolymers derived from crotonic acid, such as those comprising vinyl acetate or propionate units in their chain and optionally other monomers such as allylic esters or methallylic esters, vinyl ether and vinyl ester of an acid chosen from linear and branched saturated carboxylic acids with a long hydrocarbon chain, such as chains comprising at least 5 carbon atoms, it being possible for these polymers optionally to be grafted and crosslinked, or vinyl, allylic and methallylic esters of an α- or β-cyclic carboxylic acid. Such polymers are described, inter alia, in French Patent Nos. FR-A-1 222 944, FR-A-1 580 545, FR-A-2 265 782, FR-A-2 265 781, FR-A-1 564 110 and FR-A-2 439 798. A commercial product falling into this class is the resin 28-29-30 sold by the company National Starch.
D) copolymers derived from C₄-C₈ monounsaturated carboxylic acids or anhydrides chosen from:
   copolymers comprising (i) at least one monomeric unit chosen from maleic, fumaric, and itaconic acids and anhydrides and (ii) at least one monomeric unit chosen from vinyl esters, vinyl ethers, vinyl halides, phenylvinyl derivatives, acrylic acids and esters thereof, wherein the anhydride functions of these copolymers are optionally monoesterified or monoamidated. Such polymers are described, for example, in U.S. Pat. No. 2,047,398, U.S. Pat. No. 2,723,248, and U.S. Pat. No. 2,102,113 and GB Patent No. 839 805. Examples of commercial products include those sold under the names Gantrez AN and ES by the company ISP.
   copolymers comprising (i) at least one monomeric unit chosen from maleic, citraconic, and itaconic anhydrides and (ii) at least one monomeric unit chosen from allylic and methallylic esters optionally comprising at least one group chosen from acrylamide, methacrylamide, α-olefin, acrylic and methacrylic ester, acrylic and methacrylic acid, and vinylpyrrolidone groups in their chain, wherein the anhydride functions of these copolymers are optionally monoesterified or monoamidated.

These polymers are described, for example, in French Patent Nos. FR-A-2 350 384 and FR-A-2 357 241.
E) polyacrylamides comprising carboxylate groups; and
F) anionic polyurethanes, such as the product sold by BASF under the name Luviset PUR.

The anionic polymers comprising sulfonic groups may, for example, be chosen from polymers comprising vinylsulfonic, styrenesulfonic, naphthalenesulfonic, or acrylamidoalkylsulfonic units.

Examples of these polymers include:
polyvinylsulfonic acid salts having a molecular weight ranging from 1000 to 100 000, and copolymers with an unsaturated comonomer such as acrylic or methacrylic acids and esters thereof, as well as acrylamide and derivatives thereof, and vinyl ethers and vinylpyrrolidone;
polystyrenesulfonic acid salts such as the sodium salts sold, for example, under the name Flexan 130 by National Starch. These compounds are described in Patent No. FR-A-2 198 719; and
polyacrylamidosulfonic acid salts such as those mentioned in U.S. Pat. No. 4,128,631 and, for example, polyacrylamidoethylpropanesulfonic acid.

According to one embodiment, it is also possible to use grafted silicone anionic film-forming polymers comprising a polysiloxane portion and a portion comprising a nonsilicone organic chain, one of the two portions constituting the main chain of the polymer, wherein the other portion is grafted onto the main chain. These polymers are described, for example, in Patent Application Nos. EP-A-0 412 704, EP-A-0 412 707, EP-A-0 640 105 and WO 95/00578, EP-A-0 582 152 and WO 93/23009 and U.S. Pat. No. 4,693,935, U.S. Pat. No. 4,728,571 and U.S. Pat. No. 4,972,037.

Such polymers may, for example, be chosen from copolymers that can be obtained by radical polymerization from a monomer mixture comprising:
a) 50 to 90% by weight of tert-butyl acrylate;
b) 0 to 40% by weight of acrylic acid;
c) 5 to 40% by weight of silicone macromer of formula: 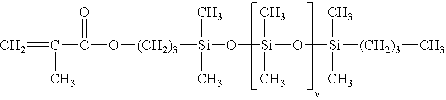
   wherein v is a number ranging from 5 to 700; wherein the weight percentages are calculated relative to the total weight of the monomers.

Other examples of grafted silicone polymers include polydimethylsiloxanes (PDMSs) onto which are grafted, via a thiopropylene connecting chain, mixed polymer units of poly(meth)acrylic acid and polyalkyl(meth)acrylate and polydimethylsiloxanes (PDMSs) onto which are grafted, via a thiopropylene connecting chain, polymer units of the polyisobutyl(meth)acrylate.

Functionalized or nonfunctionalized, silicone or nonsilicone polyurethanes may also be used as the at least one film-forming polymer.

The polyurethanes may, for example, be chosen from those disclosed in Patent Nos. EP-A-0 751 162, EP-A-0 637 600, FR-A-2 743 297 and EP-A-0 648 485, and Patent No. EP-A-0 656 021 and Patent Publication No. WO 94/03510 from the company BASF and Patent No. EP-A-0 619 111 from the company National Starch.

In one embodiment, the anionic film-forming polymers may be chosen from acrylic acid copolymers, such as acrylic acid/ethyl acrylate/N-tert-butylacrylamide terpolymers sold under the name Ultrahold Strong by the company BASF, copolymers derived from crotonic acid, such as vinyl acetate/vinyl tert-butylbenzoate/crotonic acid terpolymers and crotonic acid/vinyl acetate/vinyl neododecanoate terpolymers sold under the name Resin 28-29-30 by the company National Starch, polymers derived from maleic, fumaric or itaconic acids or anhydrides with vinyl esters, vinyl ethers, vinyl halides, phenylvinyl derivatives and acrylic acid and esters thereof, such as the methyl vinyl ether/monoesterified maleic anhydride copolymers sold, for example, under the name Gantrez by the company ISP, the copolymers of methacrylic acid and of methyl methacrylate sold under the name Eudragit L by the company Rohm Pharma, the copolymers of methacrylic acid and of ethyl acrylate sold under the name Luvimer MAEX and MAE by the company BASF, vinyl acetate/crotonic acid copolymers and vinyl acetate/crotonic acid copolymers grafted with polyethylene glycol sold under the name Aristoflex A by the company BASF, and polyurethane Luviset PUR sold by the company BASF.

In another embodiment, the anionic film-forming polymers may, for example, be chosen from methyl vinyl ether/monoesterified maleic anhydride copolymers sold under the name Gantrez ES 425 by the company ISP, acrylic acid/ethyl acrylate/N-tert-butylacrylamide terpolymers sold under the name Ultrahold Strong by the company BASF, copolymers of methacrylic acid and methyl methacrylate sold under the name Eudragit L by the company Rohm Pharma, vinyl acetate/vinyl tert-butylbenzoate/crotonic acid terpolymers and crotonic acid/vinyl acetate/vinyl neododecanoate terpolymers sold under the name Resin 28-29-30 by the company National Starch, copolymers of methacrylic acid and ethyl acrylate sold under the name Luvimer MAEX or MAE by the company BASF and vinylpyrrolidone/acrylic acid/lauryl methacrylate terpolymers sold under the name Acrylidone LM by the company ISP, and polyurethane Luviset PUR sold by the company BASF.

The amphoteric film-forming polymers may, for example, be chosen from polymers comprising units B and C distributed randomly in the polymer chain, wherein B is chosen from units derived from a monomer comprising at least one basic nitrogen atom and C is chosen from units derived from an acid monomer comprising at least one group chosen from carboxylic and sulfonic groups, or B and C, which may be identical or different, are each chosen from groups derived from carboxybetaine or sulfobetaine zwitterionic monomers;
B and C can also be chosen, for example, from cationic polymer chains comprising at least one group chosen from primary, secondary, tertiary, and quaternary amine groups, wherein at least one of the amine groups bears at least one group chosen from carboxylic and sulfonic groups connected via a hydrocarbon group or B and C form part of a chain of a polymer comprising a β-dicarboxylic ethylene unit, wherein one of the carboxylic groups has been made to react with a polyamine comprising at least one group chosen from primary and secondary amine groups

The amphoteric film-forming polymers corresponding to the definition given above may, for example, be chosen from the following polymers:
(1) polymers resulting from the copolymerization of a monomer derived from a vinyl compound bearing a carboxylic group such as, acrylic acid, methacrylic acid, maleic acid, α-chloroacrylic acid, and a basic monomer derived from a substituted vinyl compound comprising at least one basic atom, such as, dialkylaminoalkyl methacrylate and acrylate, dialkylaminoalkylmethacrylamide and -acrylamide. Such compounds are described in U.S. Pat. No. 3,836,537. Mention may also be made of the sodium acrylate/acrylamidopropyltrimethylammonium chloride copolymer sold under the name Polyquart KE 3033 by the company Henkel.

The vinyl compound may also be a dialkyldiallylammonium salt such as diethyldiallylammonium chloride. The copolymers of acrylic acid and of the latter monomer are sold under the names Merquat 280, Merquat 295, and Merquat Plus 3330 by the company Calgon.
(2) polymers comprising units derived from:
   a) at least one monomer chosen from acrylamides and methacrylamides, substituted on the nitrogen atom with at least one alkyl group,
   b) at least one acidic comonomer comprising at least one reactive carboxylic group, and
   c) at least one basic comonomer, such as esters comprising primary, secondary, tertiary and quaternary amine substituents of acrylic and methacrylic acids and the product of quaternization of dimethylaminoethyl methacrylate with dimethyl or diethyl sulfate.

The N-substituted acrylamides or methacrylamides which may be used include compounds wherein the alkyl groups comprise from 2 to 12 carbon atoms and, for example, N-ethylacrylamide, N-tert-butylacrylamide, N-tert-octylacrylamide, N-octylacrylamide, N-decylacrylamide, N-dodecylacrylamide, and corresponding methacrylamides.

The acidic comonomers may, for example, be chosen from acrylic acid, methacrylic acid, crotonic acid, itaconic acid, maleic acid, fumaric acid, and alkyl monoesters, comprising from 1 to 4 carbon atoms, of maleic, fumaric acids, or anhydrides.

The basic comonomers may, for example, be chosen from aminoethyl, butylaminoethyl, N,N′-dimethylaminoethyl, and N-tert-butylaminoethyl methacrylates.

The copolymers whose CTFA (4th edition, 1991) name is octylacrylamide/acrylates/butylaminoethyl methacrylate copolymer, such as the products sold under the name Amphomer or Lovocryl 47 by the company National Starch, may, for example, be used.
(3) crosslinked and acylated polyamino amides partially or totally derived from polyamino amides of formula III: 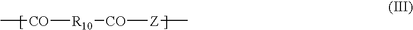
wherein R₁₀ is chosen from divalent groups derived from a saturated dicarboxylic acid; mono- and dicarboxylic aliphatic acids comprising an ethylenic double bond, esters of a lower alkanol, comprising from 1 to 6 carbon atoms, of these acids, and groups derived from the addition of any one of the acids to a bis(primary) or bis(secondary) amine; and Z is chosen from bis(primary) and mono- and bis(secondary) polyalkylenepolyamine groups and, for example, represents:
a) in proportions ranging from 60 to 100 mol %, the group IV 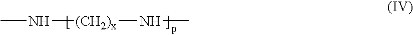
wherein x=2 and p=2 or 3, or x=3 and p=2, wherein this group is derived from a compound chosen from diethylenetriamine, triethylenetetraamine, and dipropylenetriamine;
b) in proportions ranging from 0 to 40 mol %, the group (IV) above wherein x=2 and p=1 and which is derived from ethylenediamine, or a group derived from piperazine: 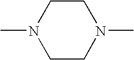
c) in proportions ranging from 0 to 20 mol %, the —NH(CH₂)₆—NH— group derived from hexamethylenediamine, these polyamino amines being crosslinked by addition reaction of a difunctional crosslinking agent chosen from epihalohydrins, diepoxides, dianhydrides, and bis-unsaturated derivatives, using from 0.025 to 0.35 mol of crosslinking agent per amine group of the polyamino amide and acylated by the action of acrylic acid, chloroacetic acid or an alkane sultone, or salts thereof.

The saturated carboxylic acids may, for example, be chosen from acids comprising 6 to 10 carbon atoms, such as adipic acid, 2,2,4-trimethyladipic acid, 2,4,4-trimethyladipic acid, terephthalic acid, acids comprising an ethylenic double bond, such as acrylic acid, methacrylic acid and itaconic acid.

The alkane sultones used in the acylation may, for example, be chosen from propane sultone and butane sultone, the salts of the acylating agents may, for example, be chosen from sodium and potassium salts.
(4) polymers comprising zwitterionic units of formula V: 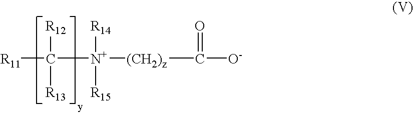
wherein R₁₁ is chosen from polymerizable unsaturated groups such as, acrylate, methacrylate, acrylamide, and methacrylamide groups, y and z are integers ranging from 1 to 3, R₁₂ and R₁₃, which may be identical or different, are each chosen from a hydrogen atom, and methyl, ethyl, and propyl groups, R₁₄ and R₁₅, which may be identical or different, are each chosen from a hydrogen atom and alkyl groups such that the sum of the carbon atoms in R₁₄ and R₁₅ do not exceed 10.

The polymers comprising such units can also comprise units derived from nonzwitterionic monomers, for example, chosen from dimethyl and diethylaminoethyl acrylate and methacrylate, alkyl acrylates and methacrylates, acrylamides and methacrylamides, and vinyl acetate.

For example, in one embodiment, a butyl methacrylate/N,N-dimethyl/carboxyaminoethylmethacrylate may be used.
(5) polymers derived from chitosan comprising monomer units corresponding to the following formulae: 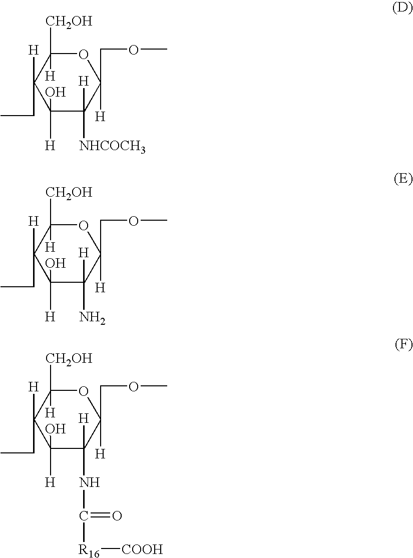
wherein: the unit D is present in proportions ranging from 0 to 30%, the unit E is present in proportions ranging from 5 to 50% and the unit F is present in proportions ranging from 30 to 90%, it being understood that, in this unit F, R₁₆ is chosen from groups of formula: 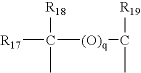
wherein, if q=0, R₁₇, R₁₈, and R₁₉, which may be identical or different, are each chosen from a hydrogen atom; methyl, hydroxyl, acetoxy, and amino groups, a monoalkylamine group, a dialkylamine group, which may be optionally interrupted by at least one nitrogen atom and/or optionally substituted with at least one functional group chosen from amine, hydroxyl, carboxyl, alkylthio, sulfonic groups and, an alkylthio group, wherein the alkyl group bears an amino functional group, wherein at least one of the groups R₁₇, R₁₈ and R₁₉ is, in this case, a hydrogen atom;
or, if q=1, R₁₇, R₁₈ and R₁₉, which may be identical or different, are each chosen from a hydrogen atom, as well as the salts formed by these compounds with bases or acids.
(6) polymers corresponding to the general formula (VI) which are described, for example, in French Patent No. FR-A-1 400 366: 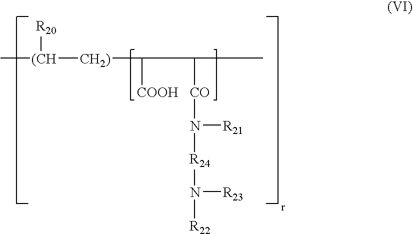
wherein:
R₂₀ is chosen from a hydrogen atom and CH₃O, CH₃CH₂O, and phenyl groups;
R₂₁ is chosen from a hydrogen atom and lower alkyl groups, such as methyl and ethyl;
R₂₂ is chosen from a hydrogen atom and lower C₁-C₆ alkyl groups, such as methyl and ethyl groups; and
R₂₃ is chosen from lower C₁-C₆ alkyl groups, such as methyl and ethyl groups and groups corresponding to the formula: —R₂₄—N(R₂₂)₂, wherein R₂₄ is chosen from —CH₂—CH₂—, —CH₂—CH₂—CH₂—, and —CH₂—CH(CH₃)— groups, and R₂₂, which may be identical or different, having the meanings mentioned above; and
r is in an integer greater or equal to 1.
(7) polymers derived from N-carboxyalkylation of chitosan, such as N-carboxymethylchitosan or N-carboxybutylchitosan.
(8) amphoteric polymers of type -D-X-D-X chosen from:
   a) polymers obtained by the action of chloroacetic acid or sodium chloroacetate on compounds comprising at least one unit of formula:
      -D-X-D-X-D- (VII)
wherein D is a radical 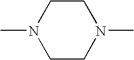
and X is chosen from symbols E or E′, wherein E and E′, which may be identical or different, are each chosen from divalent alkylene groups comprising at least one chain chosen from straight and branched chains comprising up to 7 carbon atoms in the main chain, wherein the divalent alkylene radicals are optionally substituted with at least one hydroxyl group. E or E′ can additionally comprise at least one atom chosen from oxygen, nitrogen, and sulfur atoms, and 1 to 3 rings chosen from aromatic and heterocyclic rings. The oxygen, nitrogen and sulfur atoms can being present in the form of at least one group chosen from ether, thioether, sulfoxide, sulfone, sulfonium, alkylamine, and alkenylamine groups, hydroxyl, benzylamine, amine oxide, quaternary ammonium, amide, imide, alcohol, and ester and urethane groups.
b) polymers of formula:
   -D-X-D-X— (VII′)
   wherein D is a group 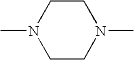
and X is chosen from symbols E or E′, and wherein at least one X is chosen from E′; E having the meaning given above and E′ is a divalent group chosen from divalent alkylene groups comprising at least one chain chosen from straight and branched chains comprising 7 carbon atoms in the main chain, wherein the divalent alkylene groups are optionally substituted with at least one hydroxyl group and comprise at least one nitrogen atom, wherein the nitrogen atom is substituted with at least one alkyl chain which is optionally interrupted by an oxygen atom and comprises at least one functional group chosen from carboxyl functional groups and hydroxyl functional groups which are betainized with at least one reactant chosen from chloroacetic acid and sodium chloroacetate.
(9) (C₁-C₅)alkyl vinyl ether/maleic anhydride copolymers partially modified by semiamidation with an N,N-dialkylaminoalkylamine, such as N,N-dimethylaminopropylamine or by semiesterification with an N,N-dialkylaminoalkanol. These copolymers can also comprise other vinyl comonomers, such as vinylcaprolactam.

In one embodiment, the amphoteric film-forming polymers are those of family (3), such as the copolymers whose CTFA name is octylacrylamide/acrylates/butylaminoethyl methacrylate copolymer, such as the products sold under the names Amphomer, Amphomer LV 71 or Lovocryl 47 by the company National Starch and those of family (4) such as the copolymers of butyl methacrylate/N,N-dimethylcarboxyaminoethylmethacrylate.

The nonionic film-forming polymers that may be used in the composition disclosed herein may be chosen, for example, from:
polyalkyloxazolines;
vinyl acetate homopolymers;
copolymers of vinyl acetate and acrylic ester;
copolymers of vinyl acetate and ethylene;
copolymers of vinyl acetate and maleic ester, for example, dibutyl maleate;
copolymers of acrylic esters, such as copolymers of alkyl acrylates and alkyl methacrylates, such as the products sold by the company Rohm & Haas under the names Primal AC-261 K and Eudragit NE 30 D, by the company BASF under the name 8845, and by the company Hoechst under the name Appretan N9212;
copolymers of acrylonitrile and a nonionic monomer chosen, for example, from butadiene and alkyl(meth)acrylates; examples of such copolymers include the products sold under the name CJ 0601 B by the company Rohm & Haas;
styrene homopolymers;
copolymers of styrene and an alkyl(meth)acrylate, such as the products Mowilith LDM 6911, Mowilith DM 611 and Mowilith LDM 6070 sold by the company Hoechst, and the products Rhodopas SD 215 and Rhodopas DS 910 sold by the company Rhône-Poulenc;
copolymers of styrene, alkyl methacrylate and alkyl acrylate;
nonionic polyurethanes;
copolymers of styrene and butadiene;
copolymers of styrene, butadiene and vinylpyridine;
copolymers of alkyl acrylate and urethane;
polyamides; and
vinyllactam homopolymers and copolymers.

In one embodiment, the alkyl groups in the nonionic polymers mentioned above may comprise 1 to 6 carbon atoms.

In one embodiment, the at least one film-forming polymer may be chosen from nonionic polymers, and, for example, nonionic polymers comprising vinyllactam units. These polymers are described, for example, in U.S. Pat. No. 3,770,683, U.S. Pat. No. 3,929,735, U.S. Pat. No. 4,521,504, U.S. Pat. No. 5,158,762 and U.S. Pat. No. 5,506,315 and in Patent Application Nos. WO 94/121148, WO 96/06592 and WO 96/10593. The non-ionic polymers may be in pulverulent form or in the form of a solution or suspension.

The homopolymers or copolymers comprising vinyllactam units may comprise units of formula: 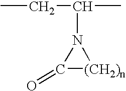
wherein n is independently 3, 4 or 5.

The number-average molar mass of the polymers comprising vinyllactam units may be greater than 5000, for example, ranging from 10 000 to 1 000 000 and, further, for example, ranging from 10 000 to 100 000.

In one embodiment, the at least one film-forming polymer that may be used may be chosen from polyvinylpyrrolidones, such as those sold under the name Luviskol K30 by the company BASF; polyvinylcaprolactams, such as those sold under the name Luviskol PLUS by the company BASF; poly(vinylpyrrolidone/vinyl acetate) copolymers, such as those sold under the name PVPVA S630L by the company ISP, Luviskol VA 73, VA 64, VA 55, VA 37 and VA 28 by the company BASF; and poly(vinylpyrrolidone/vinyl acetate/vinyl propionate) terpolymers, for example, those sold under the name Luviskol VAP 343 by the company BASF.

The at least one film-forming polymer may be present in an amount ranging from 0.01% to 20% by weight, relative to the total weight of the composition and, for example, from 0.05% to 10% by weight, relative to the total weight of the composition.

The composition disclosed herein may also comprise at least one surfactant, which may be chosen from anionic, amphoteric, nonionic, zwitterionic and cationic surfactants.

Examples of the at least one surfactant include alkyl sulfates, alkylbenzene sulfates, alkyl ether sulfates, alkyl sulfonates, quaternary ammonium salts, alkylbetaines, oxyethylenated alkylphenols, fatty acid alkanolamides, oxyethylenated fatty acid esters, and hydroxypropyl ether nonionic surfactants.

When the composition comprises at least one surfactant, it may be present in an amount less than 30% by weight and, for example, ranging from 0.5% to 10% by weight, relative to the total weight of the composition.

The composition may also comprise at least one direct dye.

The at least one direct dye may be chosen from nonionic, cationic, and anionic direct dyes.

In one embodiment, the at least one direct dye may be chosen from nitrobenzene dyes, azo, azomethine, methine, anthraquinone, naphthoquinone, benzoquinone, phenothiazine, indigoid, xanthene, phenanthridine, phthalocyanin and triarylmethane-based dyes, and natural dyes (for example, henna and camomile).

If present, the at least one direct dye may be present in an amount ranging from 0.0005% to 12% by weight, relative to the total weight of the composition, and, further, for example, from 0.005% to 6% by weight, relative to the total weight of the composition.

The composition may also comprise at least one fluorescent compound and/or at least one optical brightener.

It should be noted that the at least one fluorescent compound may, for example, be chosen from compounds that absorb light in the visible part of the spectrum and possibly in the ultraviolet region, and re-emit fluorescent light in the visible part of the spectrum, of a longer wavelength than that of the absorbed light. The wavelength of the light re-emitted by the at least one fluorescent compound may range from 500 to 650 nm.

The at least one optical brightener may be chosen from compounds that absorb light in the ultraviolet part of the spectrum, i.e., in the UVA range, at a wavelength ranging from 300 to 390 nm. These compounds re-emit fluorescent light in the visible part of the spectrum, ranging from 400 to 525 nm.

In one embodiment, the at least one fluorescent compounds and/or the at least one optical brightener that are soluble in the medium of the composition, at room temperature (ranging from 15 to 25° C.), may, for example, be used. For example, the solubility of the at least one fluorescent compound or the at least one optical brightener in the medium of the composition is greater than or equal to 0.001 g/l and, for example, greater than or equal to 0.5 g/l, at a temperature ranging from 15 to 25° C.

The at least one fluorescent compound may be chosen, for example, from substituted 4-aminophenylethenylpyridinium derivatives; naphthalimides; cationic and non-cationic coumarins; xanthenodiquinolizines; azaxanthenes; naphtholactams; azlactones; oxazines; thiazines; dioxazines, pyrenes, and nitrobenzoxadiazoles.

In addition, if they are present, the at least one fluorescent compound and the at least one optical brightener may be present in an amount ranging from 0.01% to 20% by weight, for example, from 0.05% to 10% by weight and, further, for example, from 0.1% to 5% by weight, relative to the total weight of the composition.

Moreover, the cosmetically acceptable medium of the composition may be chosen from water and mixtures of water and at least one organic solvent that is acceptable in the field.

The at least one organic solvent may be chosen from aliphatic C₂-C₄ monoalcohols, such as ethyl alcohol, isopropyl alcohol; aromatic alcohols, such as benzyl alcohol and phenylethyl alcohol; glycols and glycol ethers, such as ethylene glycol monomethyl, monoethyl and monobutyl ether, propylene glycol and ethers thereof, for example, propylene glycol monomethyl ether, butylene glycol, dipropylene glycol and diethylene glycol alkyl ethers, such as diethylene glycol monoethyl ether and monobutyl ether; and polyols, such as glycerol.

The composition may also comprise an effective amount of at least one agent chosen from those known in the art for dyeing human keratin fibers. For example, the at least one agent may be chosen from thickeners, antioxidants, fragrances, dispersants, conditioners, such as cationic and amphoteric polymers, opacifiers, UV-screening agents, preserving agents, ceramides, pseudoceramides, vitamins and provitamins, such as panthenol, and nonionic, anionic, amphoteric and cationic associative polymers.

For example, the at least one agent may be present in an amount less than or equal to 60% by weight and, for example, less than or equal to 30% by weight, relative to the total weight of the composition.

The composition disclosed herein may be provided in forms chosen from lotions, sprays, mousses, creams and gels, and any other suitable form.

In one embodiment, the composition disclosed herein is packaged under pressure in a pump-dispenser bottle or in a pressurized aerosol device.

In the case of the aerosol device, the composition may comprise at least one propellant, which may be chosen from volatile C₃-C₅ hydrocarbons, such as n-butane, propane, isobutane and pentane; chloro and fluoro hydrocarbons; and carbon dioxide, nitrous oxide, dimethyl ether, nitrogen, and compressed air.

The concentration of the propellent gas in the aerosol device depends on the nature of the at least one propellant chosen.

For example, the at least one propellant may be present in an amount ranging from 5% to 90% by weight, relative to the total weight of the composition in the aerosol device and, for example, in an amount ranging from 10% to 60% by weight, relative to the total weight of the composition in the aerosol device.

The composition disclosed herein may be applied to wet or dry keratin fibers and the fibers may then be dried or left to dry.

In one embodiment, the fibers may be shaped at the time of drying. This drying operation may takes place at a temperature ranging from 20 to 120° C. and, for example, from 20 to 80° C.

Also disclosed herein is the use of the composition as a hair product base for shaping and/or holding the hairstyle.

Other than in the example, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the disclosed composition. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the present disclosure are approximations, the numerical values set forth in the specific example is reported as precisely as possible. Any numerical value, however, inherently contain certain errors necessarily resulting from the standard deviation found in their respective testing measurements. The following example is intended to illustrate the present disclosure without limiting the scope as a result.

### EXAMPLE

Synthesis of Poly(thiophene-3-acetic acid)

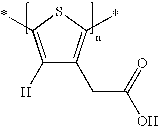

Procedure

Preparation of the Polymer: poly(ethyl thiophene-3-acetate)

25 ml of dry chloroform was introduced into a Schlenk tube under argon, the system was degassed and the following reagents were then introduced:
2.5 g of ethyl thiophene-3-acetate (14.7 mmol) 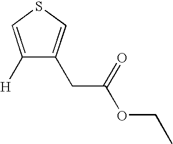
and 1 g of FeCl₃ (6.15 mmol).

The mixture was stirred for 24 hours under argon at 50° C.

The poly(ethyl thiophene-3-acetate) polymer was then precipitated in heptane. The polymer was then dissolved in a tetrahydrofuran solution.

Infrared Characterization:

C═O band: 1719 cm⁻¹; CH₂, CH₃ bands=2979 cm⁻¹, 2934 cm⁻¹ and disappearance of the CH band at 3102 cm⁻¹ present in the monomer.

Hydrolysis of the Polymer: poly(ethyl thiophene-3-acetate) to form poly(thiophene-3-acetic acid)

The polymer obtained above was then hydrolysed with an excess of 50 ml of an aqueous sodium hydroxide solution (2N) for 48 hours at 70° C., followed by acidification with concentrated HCl up to the point of precipitation of the product: poly(thiophene-3-acetic acid).

The polymer was then filtered off and washed several times with distilled water in order to remove the traces of catalyst.

Infrared Characterization of the Polymer:
C═O band: 1740 cm⁻¹; COO 1580 cm⁻¹; OH (broad band 3000-3500 cm⁻¹)

Neutralization of the poly(thiophene-3-acetic acid) polymer:

The poly(thiophene-3-acetic acid) polymer (2 g) was dissolved in tetrahydrofuran (30 g) and neutralized with a proportion of 1 mol of sodium hydroxide per mole of carboxylic acid.

Water (30 g) was then added.

The tetrahydrofuran was evaporated off.

An aqueous 6% solution of poly(thiophene-3-acetic acid) in the form of a sodium salt was thus obtained.

Formulation Comprising the Polymer and Process for Using Same:

| Poly(thiophene-3-acetic | | 4 g |
|---|---|---|
| acid) | | |
| Aminomethylpropanol qs | | pH 7 |
| Luviset PUR (from the | | 3 g |
| company BASF) | | |
| Ethyl alcohol | | 15 g |
| Water | qs | 100 g |

The formula was applied to dark hair. After a standing time of 5 minutes, drying (drying in air) was performed.

1. A composition comprising, in a cosmetically acceptable medium:
(a) at least one film-forming polymer and
(b) at least one conductive polymer.

2. The composition according to claim 1, wherein the at least one conductive polymer comprises at least one repeating unit chosen from:
anilines of formula (I) below: 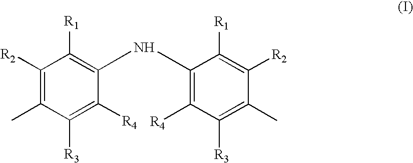
pyrroles of formulae (IIa) and (IIb) below: 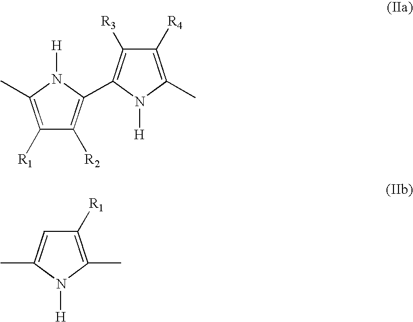
thiophenes and bisthiophenes of formulae (IIIa), (IIIb) and (IIIc) below: 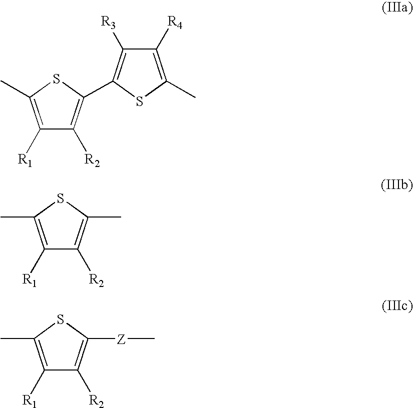
furans of formula (IV) below: 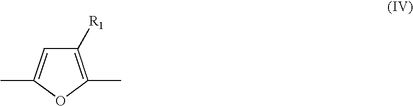
para-phenylene sulfides of formula (V) below: 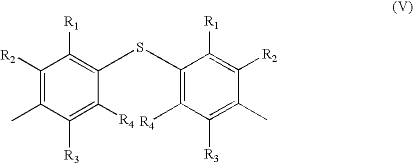
para-phenylenevinylenes of formula (VI) below: 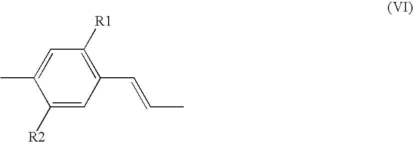
indoles of formula (VII) below: 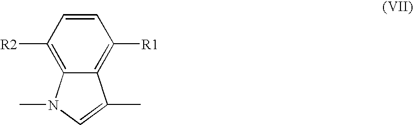
aromatic amides of formulae (VIIIa), (VIIIb), (VIIIc) and (VIIId) below: 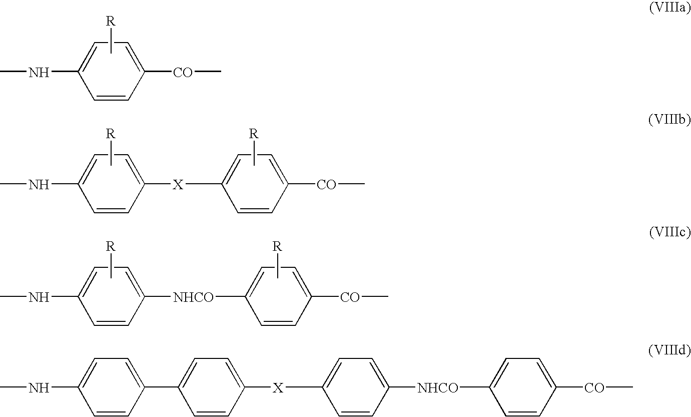
aromatic hydrazides of formulae (IXa), (IXb) and (IXc) below: 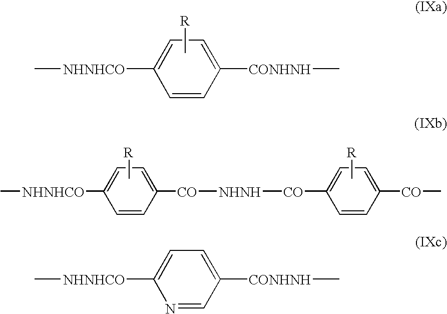
aromatic azomethines of formulae (Xa), (Xb) and (Xc) below: 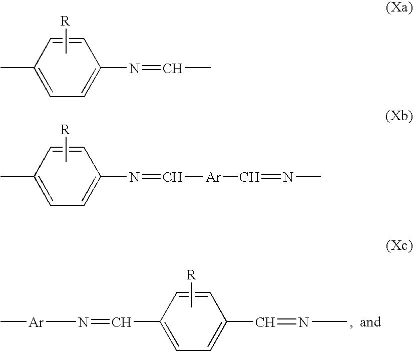
and
aromatic esters of formulae (XIa), (XIb) and (XIc) below: 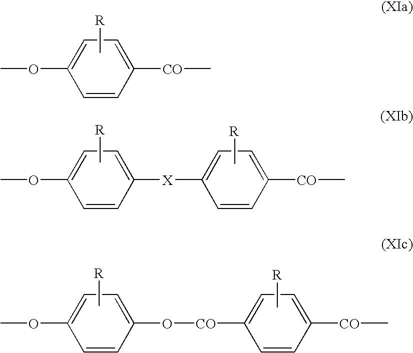
wherein, in formulae (I) to (XI):
R, R1, R2, R3, and R4, which may be identical or different, are each chosen from a hydrogen atom; —R′, —OR′, —COOR′, and —OCOR′, wherein R′ is chosen from linear and branched C₁-C₂₀ alkyl radicals; halogen atoms; nitro radicals; cyano radicals; cyanoalkyl radicals; solubilizing groups; and solubilizing groups comprising a spacer group that bonds to the ring, it being understood that at least one radical chosen from R, R1, R2, R3, and R4 is a solubilizing group;
Ar is chosen from radicals comprising at least one radical chosen from monoaromatic and polyaromatic radicals;
X is chosen from —NHCO—, —O—, —S—, —SO₂—, —N═N—, —C(CH₃)₂—, —CH₂—, —CH═CH— and —CH═N—; and
Z is chosen from —CH═CH— and —C≡C—.

3. The composition according to claim 2, wherein the solubilizing groups are chosen from:
carboxylic (—COOH) radicals and carboxylate (—COO-M⁺) radicals, wherein M is chosen from alkali metals, alkaline-earth metals, organic amines, alkanolamines, and amino acids;
sulfonic (—SO₃H) radicals and sulfonate (—SO₃⁻M⁺) radicals, wherein M is chosen from alkali metals, alkaline-earth metals, organic amines, alkanolamines, and amino acids;
primary, secondary, and tertiary amine radicals;
quaternary ammonium radicals;
hydroxyl radicals; and
poly((C₂-C₃)alkylene oxide) radicals.

4. The composition according to claim 3, wherein the quaternary ammonium radicals are chosen from —N(R′)₃⁺Z⁻ groups, wherein Z is chosen from Br, Cl, and (C₁-C₄)alkyl-OSO₃ and R′, which may be identical or different, is chosen from linear and branched C₁ to C₂₀ alkyls, or two R's form a heterocycle with the nitrogen to which they are attached.

5. The composition according to claim 2, wherein the solubilizing groups are connected to the ring via a spacer group.

6. The composition according to claim 5, wherein the spacer group is chosen from —R″—, —OR″—, —OCOR″—, and —COOR″—, wherein R″ is chosen from linear and branched C₁-C₂₀ alkyl radicals optionally comprising at least one hetero atom.

7. The composition according to claim 2, wherein R, R1, R2, R3, and R4, which may be identical or different, are each chosen from hydrogen, R′, —OR′, —OCOR′—COOR′, wherein R′ is chosen from linear and branched C₁-C₆ alkyl radicals, and from the following neutralized or non-neutralized solubilizing groups: —COOH, —CH₂COOH, —CH₂OH, —(CH₂)₆OH, —(CH₂)₃SO₃H, —O(CH₂)₃SO₃H, —O(CH₂)₃N(CH₂CH₃)₂, —[(CH₂)₂O]ₓCH₂CH₂OH, and —[(CH₂)₂O]ₓCH₂CH₂OCH₃, wherein x is an average number ranging from 0 to 200.

8. The composition according to claim 1, wherein the at least one conductive polymer is in a form that is soluble in the cosmetically acceptable medium.

9. The composition according to claim 2, wherein the at least one conductive polymer comprises at least one solubilizing group per repeating unit.

10. The composition according to claim 2, wherein the solubilizing groups are chosen from carboxylic acid radicals; neutralized carboxylic acid radicals, sulfonic acid radicals; neutralized sulfonic acid radicals; tertiary amine radicals; and quaternary ammonium radicals; wherein the solubilizing groups are optionally connected to the ring via a spacer group; and salts thereof.

11. The composition according to claim 10, wherein the quaternary ammonium radicals are chosen from —N(R′)₃⁺Z⁻ radicals, wherein Z is chosen from Br, Cl, (C₁-C₄)alkyl-OSO₃ and R′, which may be identical or different, is chosen from linear and branched C₁-C₂₀ alkyl radicals.

12. The composition according to claim 10, wherein the spacer group is chosen from C₁-C₂₀ alkyl radicals.

13. The composition according to claim 2, wherein the at least one conductive polymer comprises at least one unit chosen from units of formula (IIIa), (IIIb) and (IIIc), wherein at least one radical chosen from R1, R2, R3, and R4 of formula (IIIa) and R1 and R2 of formulae (IIIb) and (IIIc) is chosen from carboxylic acid solubilizing radicals, in neutralized or non-neutralized form, optionally connected to the ring via a spacer group, and wherein the other radical(s) is(are) hydrogen(s).

14. The composition according to claim 13, wherein the spacer group is chosen from linear or branched C₁-C₂₀ alkyl radicals.

15. The composition according to claim 1, wherein the at least one conductive polymer is present in an amount greater than or equal to 0.001% by weight, relative to the total weight of the composition.

16. The composition according to claim 15, wherein the at least one conductive polymer is present in an amount less than or equal to 50% by weight, relative to the total weight of the composition.

17. The composition according to claim 15, wherein the at least one conductive polymer is present in an amount ranging from 0.1% to 50% by weight, relative to the total weight of the composition.

18. The composition according to claim 1, wherein the at least one conductive polymer has a conductivity ranging from 1×10⁻⁵ to 5×10⁵ siemens/cm.

19. The composition according to claim 18, wherein the at least one conductive polymer has a conductivity ranging from 1×10⁻³ to 1×10⁵ siemens/cm.

20. The composition according to claim 19, wherein the at least one conductive polymer has a conductivity ranging from 1×10⁻¹ to 1×10⁴ siemens/cm.

21. The composition according to claim 1, wherein the at least one film-forming polymer is chosen from cationic, anionic, amphoteric, and nonionic polymers.

22. The composition according to claim 21, wherein the at least one film-forming polymer is an anionic film-forming polymer comprising at least one carboxylic group, chosen from:
A) acrylic or methacrylic acid homopolymers or copolymers, and salts thereof, copolymers of acrylic acid and acrylamide, and sodium salts of polyhydroxycarboxylic acids;
B) copolymers of acrylic or methacrylic acid with a monoethylenic monomer, optionally grafted onto a polyalkylene glycol, and optionally crosslinked;
C) copolymers derived from crotonic acid;
D) copolymers derived from C₄-C₈ monounsaturated carboxylic acids or anhydrides chosen from:
copolymers comprising (i) at least one monomeric unit chosen from maleic, fumaric, itaconic acids and anhydrides and (ii) at least one monomeric unit chosen from vinyl esters, vinyl ethers, vinyl halides, phenylvinyl derivatives, acrylic acid and esters thereof, wherein the anhydride functions of these copolymers are optionally monoesterified or monoamidated;
copolymers comprising (i) at least one monomeric unit chosen from maleic, citraconic, and itaconic anhydride units and (ii) at least one monomeric unit chosen from allylic and methallylic esters, optionally comprising at least one group chosen from acrylamide, methacrylamide, α-olefin, acrylic ester, methacrylic ester, acrylic acid, methacrylic acid, and vinylpyrrolidone groups in their chain;
E) polyacrylamides comprising carboxylate groups;
F) anionic polyurethanes; and
G) grafted silicone polymers comprising a polysiloxane portion and a portion comprising a non-silicone organic chain, one of the two portions constituting the main chain of the polymer, wherein the other portion is grafted onto the main chain.

23. The composition according to claim 22, wherein the monoethylenic monomer, of the copolymers of acrylic or methacrylic acid, is chosen from ethylene, styrene, vinyl esters, and acrylic and methacrylic acid esters.

24. The composition according to claim 22, wherein the polyalkylene glycol, of the copolymers of acylic or methacrylic acid with a monoethylenic monomer, is a polyethylene glycol.

25. The composition according to claim 21, wherein the at least one film-forming polymer is a nonionic polymer chosen from:
polyalkyloxazolines;
vinyl acetate homopolymers;
copolymers of vinyl acetate and of acrylic ester;
copolymers of vinyl acetate and of ethylene;
copolymers of vinyl acetate and of maleic ester;
acrylic ester copolymers;
copolymers of acrylonitrile and a nonionic monomer;
styrene homopolymers;
copolymers of styrene and alkyl(meth)acrylate;
copolymers of styrene, alkyl methacrylate, and alkyl acrylate;
nonionic polyurethanes;
copolymers of styrene and butadiene;
copolymers of styrene, butadiene, and vinylpyridine;
copolymers of alkyl acrylate and urethane;
polyamides; and
vinyllactam homopolymers and copolymers.

26. The composition according to claim 25, wherein the copolymers of vinyl acetate and maleic ester is a dibutyl maleate.

27. The composition according to claim 1, wherein the at least one film-forming polymer is present in an amount ranging from 0.01% to 20% by weight, relative to the total weight of the composition.

28. The composition according to claim 27, wherein the at least one film-forming polymer is present in an amount ranging from 0.05% to 10% by weight, relative to the total weight of the composition.

29. The composition according to claim 1, wherein the cosmetically acceptable medium is chosen from water and mixtures of water and at least one organic solvent.

30. The composition according to claim 29, wherein the at least one organic solvent is chosen from C₂-C₄ aliphatic monoalcohols, aromatic alcohols, glycols, glycol ethers, and polyols.

31. The composition according to claim 1, further comprises at least one direct dye chosen from nonionic, cationic, and amphoteric direct dyes.

32. The composition according to claim 31, wherein the at least one direct dye is chosen from nitrobenzene dyes, azo dyes, anthraquinone, naphthoquinone and benzoquinone dyes, indigoid dyes, triarylmethane-based dyes, and natural dyes.

33. The composition according to claim 31, wherein the at least one direct dye is present in an amount ranging from 0.0005% to 12% by weight, relative to the total weight of the composition.

34. The composition according to claim 1, wherein the composition comprises at least one fluorescent compound.

35. The composition according to claim 34, wherein the at least one fluorescent compound is chosen from substituted 4-aminophenylethenylpyridinium derivatives; naphthalimides; cationic and non-cationic coumarins; xanthenodiquinolizines; azaxanthenes; naphtholactams; azlactones; oxazines; thiazines; dioxazines; pyrenes; and nitrobenzoxadiazoles.

36. The composition according to claim 1, wherein the composition comprises at least one optical brightener.

37. The composition according to claim 36, wherein the at least one optical brightener is chosen from stilbene derivatives, coumarin derivatives, oxazole and benzoxazole derivatives, and imidazole derivatives.

38. The composition according to claim 34, wherein the at least one fluorescent compound is present in an amount ranging from 0.01% to 20% by weight, relative to the total weight of the composition.

39. The composition according to claim 36, wherein the at least one optical brightener is present in an amount ranging from 0.01% to 20% by weight, relative to the total weight of the composition.

40. The composition according to claim 1, further comprising at least one propellant.

41. The composition according to claim 40, wherein the at least one propellant is present in an amount ranging from 5% to 90% by weight, relative to the total weight of the composition.

42. The composition according to claim 41, wherein the at least one propellant is present in an amount ranging from 10% to 60% by weight, relative to the total weight of the composition.

43. A process for treating human keratin fibers, comprising,
applying, to wet or dry fibers, at least one composition comprising, in a cosmetically acceptable medium:
(a) at least one film-forming polymer and
(b) at least one conductive polymer; and
drying the fibers or leaving the fibers to dry.

44. The process according to claim 43, wherein the human keratin fibers are hair.

45. A hair product base comprising, in a cosmetically acceptable medium,
(a) at least one film-forming polymer and
(b) at least one conductive polymer, wherein the hair product base is effective for shaping and/or holding hair.

46. A device comprising, in a cosmetically acceptable medium:
(a) at least one film-forming polymer and
(b) at least one conductive polymer.

47. A method for imparting at least one optical effect on keratin fibers comprising applying to the fibers, at least one composition comprising, in a cosmetically acceptable medium, at least one conductive polymer and at least one fixing polymer.
